(19) United States
(12) Patent Application Publication
(10) Pub. No.: US 2004/0215238 A1
(43) Pub. Date: Oct. 28, 2004

(54) Pacemaker with improved capability for detecting onset of tachyarrhythmias and heart failure

(76) Inventors: Peter M. van Dam, Arnhem (NL); Adriaan van Oosterom, Linden (NL)

Correspondence Address: MEDTRONIC, INC., 710 MEDTRONIC PARKWAY NE, MS-LC340, MINNEAPOLIS, MN, 55432-5604, US

(21) Appl. No.: 10/422,712

(22) Filed: Apr. 24, 2003

(51) Int. Cl. A61N 1/365

(52) U.S. Cl. 607/4

(57) ABSTRACT

The invention provides a system and method for determining cardiac condition by examining the width of cardiac excitation waves sensed by a tip electrode. In one embodiment, an atrial lead with a tip electrode is positioned against the inside of the atrial wall, and senses passing depolarization (P-wave) waves. The sensed signals are processed to determine effective duration, and then analyzed to determine if there has been a change in conduction velocity. A predetermined decrease in conduction velocity is interpreted to mean that the atrium is at the onset of AF and appropriate atrial pacing therapy is initiated. In a second embodiment, a lead with a tip electrode is positioned adjacent to the left free ventricular wall and R wave durations are obtained. For substantially constant ventricular conduction conditions, R wave duration variations are analyzed to determine ventricular wall thickening of a degree that indicates onset of chronic heart failure.

### FIELD OF THE INVENTION

This invention relates to pacing systems that incorporate a diagnostic capability for monitoring cardiac conditions and providing a response to a detected abnormal condition and, more particularly, to implantable pacing systems and methods for monitoring cardiac conditions and sensing the onset of atrial tachyarrhythmias or heart failure, and that provide a pacing therapy in response to such sensing.

### BACKGROUND OF THE INVENTION

Previously, the treatment of abnormal heart rhythms, or cardiac arrhythmias, has received a great deal of attention and has been the subject of much investigation and medical literature.

One of the most common such abnormal heart rhythm is atrial fibrillation, also referred to as AF, which affects over 2 million persons every year. AF is characterized by an uncontrolled high rate shivering or quivering of the atria, with the atria fibrillating at 300 to 600 times per minute. During AF there is a loss of synchrony between the atria and the ventricles, resulting in substantial failure of pumping from the atria to the ventricles. The passage of excitation impulses through the AV node to the ventricles is irregular and at an abnormally high rate, resulting in irregular ventricular rates on the order of 150 beats per minute and above. Consequently, when AF becomes symptomatic, the patient feels shortness of breath and dizziness, reflecting the failure of good pumping function.

AF presents more serious consequences to the patient than just the symptoms, which themselves can be debilitating. During AF the blood is not efficiently flushed from the heart's chambers, resulting in pooling and a greater likelihood of clotting. In about 5% of AF cases, such clots are discharged from the heart and produce a stroke; the American Heart Association estimates that AF causes over 70,000 strokes annually. And AF can lead to other problems, including cardiomyopathy and congestive heart failure (CHF). AF can occur directly, or it can occur as a secondary event initiated from other arrhythmias such as atrial tachycardia (AT) or atrial flutter.

The natural activation of the atrial myocardium originates from the sinus node and travels throughout the entire atrium, causing contraction. FIG. 6 illustrates the position of an activation, or depolarization wavefront travelling through atria. Since the atrial wall is rather thin, about 2 mm, this means that approximately all the myocardial cells through the atrial wall at the wavefront are activated simultaneously. A tip electrode positioned against the atrial wall senses when a depolarization wave travels past the electrode, such that the intracardial P-wave is a local phenomenon. The repolarization signal is too weak to be normally seen, such that the P-wave that can be sensed by such a pacemaker lead is a signature of the depolarization wave as it passes the electrode. The maximum amplitude and “width” of the P-wave portion of the iecg signal are a function of the velocity of the activation front as it passes the electrode, and the smaller the electrode the greater the spike of the sensed signal.

The medical literature shows that the atria are vulnerable to AT or AF when the atrial wall is characterized by non-uniformity of conduction and by slow conduction, giving rise to reentry circuits or pathways. Slow impulse conduction can give rise to reentrant arrhythmias by reducing the effective wavelength of the reentering wavefront, such that the available reentry pathway can accommodate it. Stating the matter differently, a slower conduction enables a return or circular path with the proper timing such that when the wavefront has made a loop the initially depolarized cells have repolarized and are no longer refractory. In such a case, the cells can be depolarized by the reentry wave, triggering a continuing impulse wavefront without receiving an impulse from the sinus node. A number of factors can determine the velocity at which an action potential, or impulse is propagated. Among these are the fast inward sodium current during the upstroke of the action potential and the axial resistance (degree of cellular coupling) to the flow of the local current through the myocardial fibres. The conduction property of cardiac muscle is anisotropic in that it conducts longitudinally axially, or longitudinally better than it does transversely. A healthy heart has uniform anistropy, such that the advancing wavefront is smooth in all directions. A heart characterized by non-uniform anistropy is one where the wavefront does not advance smoothly, and includes areas of unidirectional block and return conduction pathways. It is this non-uniform anisotropy, together with slower conduction, that can produce an arrhythmogenic substrate and make the heart vulnerable to AF.

By monitoring the effective conduction velocity of the myocardium, the development of an arrhythmogenic substrate can be determined, enabling an indication of the onset of AF or another arrhythmia. When the velocity drops, this indicates either a slowing of conduction or a widening (thickening) of the heart wall. In the case of the atria, the heart wall retains a rather constant thickness, so a sensed decrease in conduction velocity indicates a slowing of conduction. When the conduction velocity is determined to have dropped to a certain threshold, or a given amount below the patient's normal velocity, this is an indication of conditions that give rise to AF.

Our studies of conduction in the human heart wall show that the waveform of an impulse that propagates through cardiac tissue can provide information concerning conduction velocity, given that the wall thickness is constant. The conduction velocity and wave width are closely related for an atrial activation wave. In an atrium, the wall is rather thin, being on average about 2 mm in thickness. The activation wave in a normal atrium takes about 125 ms, i.e., this is the time from initiation of the wave at the sinus node to finished activation of the excitable atrial muscle. Assuming no change in wall thickness, a change in the speed of passage of the atrial waveform is an indication of a change in effective conduction velocity, and vice versa.

We have examined the effect of wall thickness on an iegm waveform measured on the tip electrode 21 of a lead positioned against a heart wall 8 as illustrated in FIG. 7A. We assume that the impulse wave passes perpendicularly to the tip, and that the impulse wave is a flat wave with a width w (corresponding to the wall thickness), as shown in FIG. 7A. For this situation, the theoretical sensed waveform at the tip located at x(o) is as shown in FIG. 7B. It is noted that the potential at the tip is only half the maximum value when the excitation wave is a distance w away from the tip. From this we conclude that for constant propagation velocity, a wider wall thickness will induce a wider waveform (longer pulse width in terms of time, Δt) in an electrode placed against the heart wall.

Our studies have demonstrated that in the atria, wall thickness is substantially uniform. However, this is not the case for the left ventricle of a patient who is progressing toward heart failure (HF), commonly also referred to as congestive heart failure (CHF). In CHF, a first response of the heart to increased load is to increase its contraction power, i.e., ability to pump more blood. In many cases this means that the heart wall becomes thicker, or hypertrophied. When this happens, the stroke volume reduces, which eventually means that the patient becomes short of energy. The last stage of CHF can be a dilated heart, a condition that occurs due to apoptosis, i.e., cell death. In this condition, the heart wall becomes thinner, with even more decreased pumping strength. The left ventricle is the chamber that pumps blood into the body, and therefore the one that must be monitored for changes in wall thickness. Assuming that conduction velocity throughout the left ventricle is substantially constant at onset of CHF and through its progression, the wall thickness can be detected by an electrode positioned just beneath the left free wall. The greater the width (duration) of the sensed signal from passage of the activation wave (the R wave), the greater the wall thickness, and vice versa.

The pacing art has produced a number of pacing therapies that are available to aid in suppressing AF or alleviating the symptoms of AF. Thus, pacemaker systems have incorporated therapies that respond to high atrial rates, designed to treat already developed arrhythmias. However, what is needed is a better means for early detection of the onset of AF. Such early detection or prediction of AF can be accomplished by getting an accurate picture of a developing arrhythmic substrate, which in turn can be obtained from sensed waveform data. In U.S. Pat. No. 5,184,615 to Nappholz, the pacemaker obtains a paced depolarization integral (PDI) that is the time integral of the depolarization voltage amplitude over the interval of the QRS complex. This integral is analyzed over time, to detect changes that might indicate cardiac arrhythmias. However, the integral is not an accurate indication of conduction changes; and a measure of a QRS waveform in response to a pacing pulse does not provide any predictability of onset of AF. The approach taken in U.S. Pat. No. 5,243,981 is an improvement, but still has problems. In that patent, two spaced electrodes are positioned against the heart wall and coupled to respective amplifiers to detect the relative arrival times of the depolarization wavefront at the electrodes. The difference in arrival times is used to determine a measure of conduction velocity. However, such arrival times do not contain information concerning the overall non-uniform anisotropy of the atrial wall, or the degree to which there is slowing of conduction in reentry paths that can be a sign of AF. Depending on how the wave has traveled, the excitation wave can arrive at the two points at much the same time, or with a time difference that doesn't accurately reflect the overall conduction scheme. Since the conduction changes can occur anywhere throughout the entire atria, the measurement must represent the entire wave, not just the leading edge of the wave at several points.

Publications that explain the mechanism of conduction in cardiac muscle and refer to conduction velocity include Cellular Electrophysiology, Part III, page 148 (fill in date); Arrhythmia Mecha, Part VIII, pp 490, 512 and 548-550 (fill in date); and Circulation, Chow et. al, Wavefront Propagation in Circuits Causing Human VT, pp 2172-2178, May 2002. The following patents disclose implantable pacing systems and refer directly or indirectly to cardiac conduction velocity.

TABLE 1

| Pat. No. | Inventor(s) | Issue Date |
|---|---|---|
| 5,184,615 | Nappholz et. a. | Feb. 9, 1993 |
| 5,243,981 | Hudrlik | Sep. 14, 1993 |
| 5,267,560 | Cohen | Dec. 7, 1993 |

All patents listed in Table 1 are hereby incorporated by reference in their respective entireties. As those of ordinary skill in the art will appreciate readily upon reading the Summary of the Invention, Detailed Description of the Preferred Embodiments and Claims set forth below, many of the devices and methods disclosed in the patents of Table 1 may be modified advantageously by using the teachings of the present invention.

### BRIEF SUMMARY OF THE INVENTION

It is an object of the invention to provide a system and method applied to a heart for obtaining information from iecg signals and deriving therefrom data that represents a measure of overall atrial conduction velocity, and for processing the velocity data to obtain an indication of whether or not the heart is at or near AF.

It is another object of this invention to provide an implantable pacing system with monitoring capability to obtain data indicative of conduction velocity throughout a patient's atria, and with an algorithm for processing such data to provide a determination of whether the patient is at or near AF.

It is another object of this invention to provide a method of obtaining iecg data from within a patient's atrium and for utilizing this data to provide an indication of whether the patient is at onset of AF.

It is another object of this invention to provide a system and method for determining wall thickness in the left free ventricular wall.

It is another object of this invention to provide a pacing system and method for diagnosing ventricular wall thickness based on variations in the duration of the excitation wave as it passes a location in the left free ventricular wall and utilizing such diagnosed wall thickness to obtain an indication of CHF.

It is another object of this invention to provide a system and method for determining conduction velocity in a patient's atria by utilizing a tip electrode that can be placed substantially anywhere against the wall of an atrial chamber of the patient in order to obtain local iecg signals that contain information about atrial conduction velocity.

In a first embodiment, there is provided an implantable pacing system that includes a lead having at least a tip electrode adapted to be positioned within the patient's right atrium against the cardiac wall. The sensed P-wave signals from the patient's atria are processed to obtain measures of overall atrial conduction velocity, and such measures are processed with an algorithm to provide a prediction of AF. In a preferred embodiment, the P-wave signals are processed periodically to provide an average relative velocity measure, and the measure is compared with prior stored velocity data to determine whether conduction velocity has slowed such that the patient is likely at or near AF. When and if there is an indication of the onset of AF, a suitable pacing therapy is applied.

In the practice of the method of the first embodiment, a lead with a tip electrode is inserted into the patient's right atrium and the tip electrode is positioned adjacent the atrial wall. The exact placement is not critical so long as the tip is snug with the wall muscle. Sensing of P-waves can be done either unilaterally, with the pacemaker can used as the other electrode, or bilaterally with a suitable ring electrode placed sufficiently proximal to the tip electrode so that the sensed signal is much the same as with the unilateral embodiment. The P-waves are analyzed to determine their time width (duration), and each width determination in milliseconds is compared with prior values and stored. When the comparison indicates onset of AF, the pacing system is controlled to apply a suitable pacing therapy.

In a second embodiment of the invention, a lead is placed against or near the left ventricular free wall, e.g., within the left main coronary artery. QRS signals are obtained from the tip electrode of the lead, and width data of each such waveform is obtained. Another lead may be placed suitably in the right ventricle for obtaining reference waveforms passing a tip electrode at the end of the lead in the right ventricle, to indicate substantially constant ventricular conduction velocity. The system algorithm determines when the depolarization waves sensed in the left ventricle are stable, and when this situation is present analyzes the data from the ventricular free wall to obtain a measure of ventricular wall thickness. The ventricular wall thickness data in turn is processed to determine the degree, if any, of ventricular wall hypertrophy and CHF.

In another embodiment, plural leads are utilized as described above in a multi-chamber system and method to obtain predictions of AF, VF and/or HF.

### BRIEF DESCRIPTION OF THE DRAWINGS

FIG. 1 shows a simplified schematic view of one embodiment of an implantable medical device that can be employed in the present invention.

FIG. 2 shows a graphic representation of an implantable medical device interconnected with a human or mammalian heart, illustrating the device connector portion and the leads between the device and the heart.

FIG. 3 shows a functional schematic diagram showing the primary constituent components of an implantable medical device in accordance with an embodiment of this invention.

FIG. 4 shows a graphic representation of an embodiment of this invention showing an implantable PCD device interconnected with a heart, the system of this embodiment providing pacing, cardio version and defibrillation.

FIG. 5 is a functional schematic diagram of an implantable PCD embodiment of this invention.

FIG. 6 shows a schematic sketch of atria, with a jagged line that illustrates an advancing excitation wavefront.

FIG. 7A is a sketch that shows a depolarization wavefront passing through a heart wall with a thickness w, with a tip electrode positioned normal to the wall and the passing wavefront.

FIG. 7B is a graph of sensed potential vs. distance obtained from a tip electrode placed under a heart wall such that the depolarization impulse travels perpendicularly past the tip.

FIG. 8 is an overall flow diagram illustrating the collection of P-wave data from a tip electrode positioned against the atrial wall and the processing of such data for determining when there is an indication of the onset of AF.

FIG. 9 is an expanded sub-routine of the steps of block 202 in FIG. 8, for checking sensed waveforms and obtaining values of wave width (representing relative velocity in FIG. 8).

FIG. 10 is an expanded flow diagram of block 204 in FIG. 8, for obtaining measures of average velocity and threshold velocity.

FIG. 11A is a schematic drawing that shows a tip electrode positioned on the lateral wall of the left ventricle.

FIG. 11B is a flow diagram illustrating an algorithm for determining wall thickness in the free left ventricular wall in accordance with this invention.

FIG. 11C is a flow diagram illustrating a modification of the algorithm of FIG. 11B by using right ventricular signals as reference signals for checking for stability of ventricular conduction.

### DETAILED DESCRIPTION OF THE INVENTION

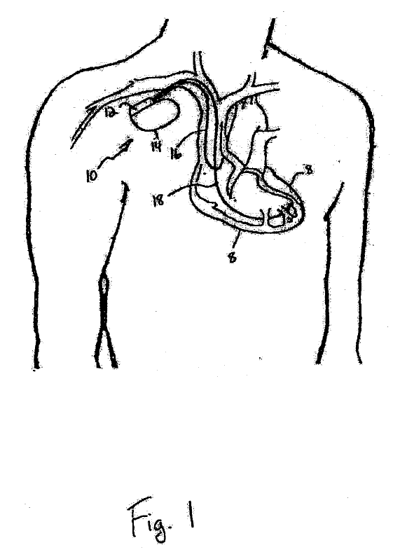

The invention may be embodied in many forms, as the following specification and Figures illustrate. FIG. 1 is a simplified schematic view of one embodiment of implantable medical device (“IMD”) 10 of the present invention. IMD 10 shown in FIG. 1 is a pacemaker comprising at least one of pacing and sensing leads 16 and 18 attached to hermetically sealed enclosure 14 and implanted near human or mammalian heart 8. Pacing and sensing leads 16 and 18 sense electrical signals attendant to the depolarization and re-polarization of the heart 8, and further provide pacing pulses for causing depolarization of cardiac tissue in the vicinity of the distal ends thereof. Leads 16 and 18 may have unipolar or bipolar electrodes disposed thereon, as is well known in the art. In the practice of this invention, as discussed further below, a unipolar type lead with a tip electrode is preferred. However, a lead with a tip electrode and a second electrode placed substantially proximal so as to get substantially equivalent unipolar tip sensing can also be used Examples of IMD 10 include implantable cardiac pacemakers disclosed in U.S. Pat. No. 5,158,078 to Bennett et al., U.S. Pat. No. 5,312,453 to Shelton et al. or U.S. Pat. No. 5,144,949 to Olson, all hereby incorporated by reference herein, each in its respective entirety.

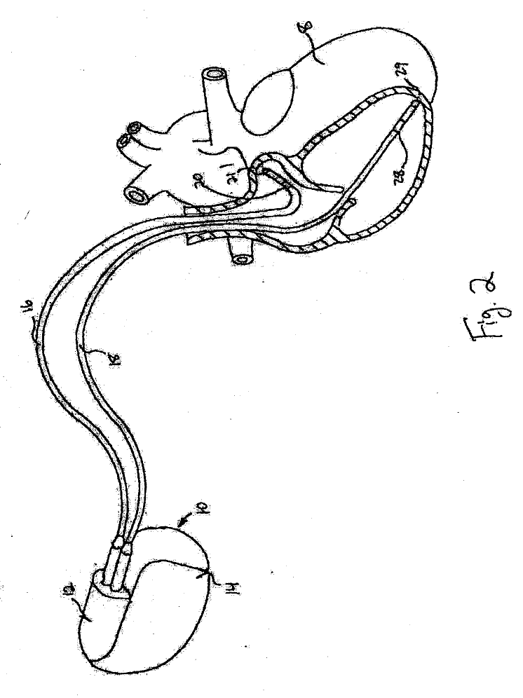

FIG. 2 shows connector module 12 and hermetically sealed enclosure 14 of IMD 10 located in and near human or mammalian heart 8. Atrial and ventricular pacing leads 16 and 18 extend from connector header module 12 to the right atrium and ventricle, respectively, of heart 8. Atrial electrodes 20 and 21 disposed at the distal end of atrial pacing lead 16 are located in the right atrium. Ventricular electrodes 28 and 29 at the distal end of ventricular pacing lead 18 are located in the right ventricle.

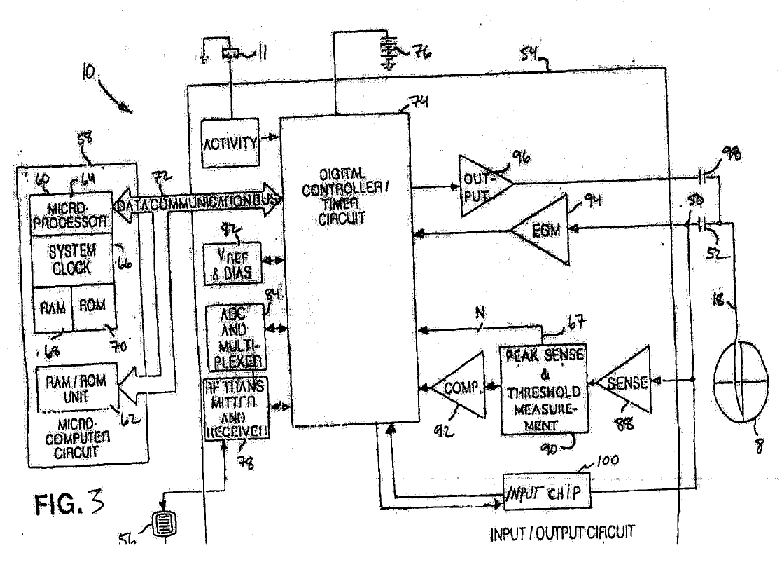

FIG. 3 shows a block diagram illustrating the constituent components of IMD 10 in accordance with one embodiment of the present invention, where IMD 10 is pacemaker having a microprocessor-based architecture. IMD 10 is shown as including activity sensor or accelerometer 11, which is preferably a piezoceramic accelerometer bonded to a hybrid circuit located inside enclosure 14. Activity sensor 11 typically (although not necessarily) provides a sensor output that varies as a function of a measured parameter relating to a patient's metabolic requirements. For the sake of convenience, IMD 10 in FIG. 3 is shown with lead 18 only connected thereto; similar circuitry and connections not explicitly shown in FIG. 3 apply to lead 16.

IMD 10 in FIG. 3 is most preferably programmable by means of an external programming unit (not shown in the Figures). One such programmer is the commercially available Medtronic Model 9790 programmer, which is microprocessor-based and provides a series of encoded signals to IMD 10, typically through a programming head which transmits or telemeters radio-frequency (RF) encoded signals to IMD 10. Such a telemetry system is described in U.S. Pat. No. 5,312,453 to Wyborny et al., hereby incorporated by reference wherein in its entirety. The programming methodology disclosed in Wyborny et al.'s '453 patent is identified herein for illustrative purposes only. Any of a number of suitable programming and telemetry methodologies known in the art may be employed so long as the desired information is transmitted to and from the pacemaker.

As shown in FIG. 3, lead 18 is coupled to node 50 in IMD 10 through input capacitor 52. Activity sensor or accelerometer 11 is most preferably attached to a hybrid circuit located inside hermetically sealed enclosure 14 of IMD 10. The output signal provided by activity sensor 11 is coupled to input/output circuit 54. Input/output circuit 54 contains analog circuits for interfacing to heart 8, activity sensor 11, antenna 56 and circuits for the application of stimulating pulses to heart 8. The rate of heart 8 is controlled by software-implemented algorithms stored in microcomputer circuit 58.

Microcomputer circuit 58 preferably comprises on-board circuit 60 and off-board circuit 62. Circuit 58 may correspond to a microcomputer circuit disclosed in U.S. Pat. No. 5,312,453 to Shelton et al., hereby incorporated by reference herein in its entirety. On-board circuit 60 preferably includes microprocessor 64, system clock circuit 66 and on-board RAM 68 and ROM 70. Off-board circuit 62 preferably comprises a RAM/ROM unit. On-board circuit 60 and off-board circuit 62 are each coupled by data communication bus 72 to digital controller/timer circuit 74. Microcomputer circuit 58 may comprise a custom integrated circuit device augmented by standard RAM/ROM components.

Electrical components shown in FIG. 3 are powered by an appropriate implantable battery power source 76 in accordance with common practice in the art. For the sake of clarity, the coupling of battery power to the various components of IMD 10 is not shown in the Figures. Antenna 56 is connected to input/output circuit 54 to permit uplink/downlink telemetry through RF transmitter and receiver telemetry unit 78. By way of example, telemetry unit 78 may correspond to that disclosed in U.S. Pat. No. 4,566,063 issued to Thompson et al., hereby incorporated by reference herein in its entirety, or to that disclosed in the above-referenced '453 patent to Wyborny et al. It is generally preferred that the particular programming and telemetry scheme selected permit the entry and storage of cardiac rate-response parameters. The specific embodiments of antenna 56, input/output circuit 54 and telemetry unit 78 presented herein are shown for illustrative purposes only, and are not intended to limit the scope of the present invention.

Continuing to refer to FIG. 3, VREF and Bias circuit 82 most preferably generates stable voltage reference and bias currents for analog circuits included in input/output circuit 54. Analog-to-digital converter (ADC) and multiplexer unit 84 digitizes analog signals and voltages to provide “real-time” telemetry intracardiac signals and battery end-of-life (EOL) replacement functions. Operating commands for controlling the timing of IMD 10 are coupled by data bus 72 to digital controller/timer circuit 74, where digital timers and counters establish the overall escape interval of the IMD 10 as well as various refractory, blanking and other timing windows for controlling the operation of peripheral components disposed within input/output circuit 54.

Digital controller/timer circuit 74 is preferably coupled to sensing circuitry, including sense amplifier 88, peak sense and threshold measurement unit 90 and comparator/threshold detector 92. Circuit 74 is further preferably coupled to electrogram (EGM) amplifier 94 for receiving amplified and processed signals sensed by lead 18. Sense amplifier 88 amplifies sensed electrical cardiac signals and provides an amplified signal to peak sense and threshold measurement circuitry 90, which in turn provides an indication of peak sensed voltages and measured sense amplifier threshold voltages on multiple conductor signal path 67 to digital controller/timer circuit 74. An amplified sense amplifier signal is then provided to comparator/threshold detector 92. By way of example, sense amplifier 88 may correspond to that disclosed in U.S. Pat. No. 4,379,459 to Stein, hereby incorporated by reference herein in its entirety.

The electrogram signal provided by EGM amplifier 94 is employed when IMD 10 is being interrogated by an external programmer to transmit a representation of a cardiac analog electrogram. See, for example, U.S. Pat. No. 4,556,063 to Thompson et al., hereby incorporated by reference herein in its entirety. Output pulse generator 96 provides pacing stimuli to patient's heart 8 through coupling capacitor 98 in response to a pacing trigger signal provided by digital controller/timer circuit 74 each time the escape interval times out, an externally transmitted pacing command is received or in response to other stored commands as is well known in the pacing art. By way of example, output amplifier 96 may correspond generally to an output amplifier disclosed in U.S. Pat. No. 4,476,868 to Thompson, hereby incorporated by reference herein in its entirety.

The specific embodiments of input amplifier 88, output amplifier 96 and EGM amplifier 94 identified herein are presented for illustrative purposes only, and are not intended to be limiting in respect of the scope of the present invention. In another embodiment, signals from the patient's heart are coupled to an input channel chip shown at 100, which chip provides outputs to the controller 74. The preferred embodiment of this chip incorporates DSP circuitry as is further disclosed in U.S. Pat. No. 6,029,087, incorporated herein by reference. The specific embodiments of such circuits may not be critical to practicing some embodiments of the present invention so long as they provide means for generating a stimulating pulse and are capable of providing signals indicative of natural or stimulated contractions of heart 8.

In some preferred embodiments of the present invention, IMD 10 may operate in various non-rate-responsive modes, including, but not limited to, DDD, DDI, VVI, VOO and VVT modes. In other preferred embodiments of the present invention, IMD 10 may operate in various rate-responsive, including, but not limited to, DDDR, DDIR, VVIR, VOOR and VVTR modes. Some embodiments of the present invention are capable of operating in both non-rate-responsive and rate responsive modes. Moreover, in various embodiments of the present invention IMD 10 may be programmably configured to operate so that it varies the rate at which it delivers stimulating pulses to heart 8 only in response to one or more selected sensor outputs being generated. Numerous pacemaker features and functions not explicitly mentioned herein may be incorporated into IMD 10 while remaining within the scope of the present invention.

The present invention is not limited in scope to single-sensor or dual-sensor pacemakers, and is not limited to IMD's comprising activity or pressure sensors only. Nor is the present invention limited in scope to single-chamber pacemakers, single-chamber leads for pacemakers or single-sensor or dual-sensor leads for pacemakers. Thus, various embodiments of the present invention may be practiced in conjunction with more than two leads or with multiple-chamber pacemakers, for example. At least some embodiments of the present invention may be applied equally well in the contexts of single-, dual-, triple- or quadruple-chamber pacemakers or other types of IMD's. See, for example, U.S. Pat. No. 5,800,465 to Thompson et al., hereby incorporated by reference herein in its entirety, as are all U.S. Patents referenced therein.

IMD 10 may also be a pacemaker-cardioverter-defibrillator (“PCD”) corresponding to any of numerous commercially available implantable PCD's. Various embodiments of the present invention may be practiced in conjunction with PCD's such as those disclosed in U.S. Pat. No. 5,545,186 to Olson et al., U.S. Pat. No. 5,354,316 to Keimel, U.S. Pat. No. 5,314,430 to Bardy, U.S. Pat. No. 5,131,388 to Pless and U.S. Pat. No. 4,821,723 to Baker et al., all hereby incorporated by reference herein, each in its respective entirety.

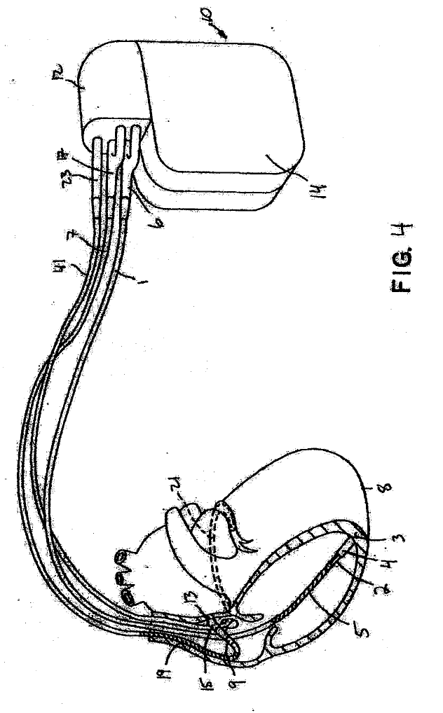

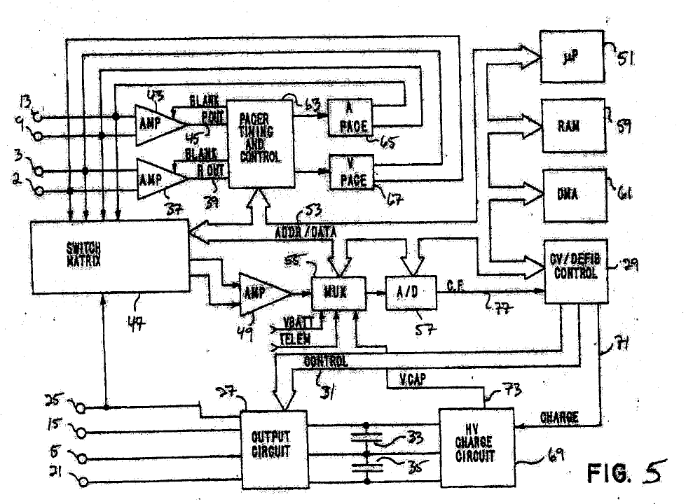

FIGS. 4 and 5 illustrate one embodiment of IMD 10 and a corresponding lead set, where IMD 10 is a PCD. In FIG. 4, the ventricular lead takes the form of leads disclosed in U.S. Pat. Nos. 5,099,838 and 5,314,430 to Bardy, and includes an elongated insulative lead body 1 carrying three concentric coiled conductors separated from one another by tubular insulative sheaths. Located adjacent the distal end of lead 1 are ring electrode 2, extendable helix electrode 3 mounted retractably within insulative electrode head 4 and elongated coil electrode 5. Each of the electrodes is coupled to one of the coiled conductors within lead body 1. Electrodes 2 and 3 are employed for cardiac pacing and for sensing ventricular depolarizations. At the proximal end of the lead is bifurcated connector 6 which carries three electrical connectors, each coupled to one of the coiled conductors. Defibrillation electrode 5 may be fabricated from platinum, platinum alloy or other materials known to be usable in implantable defibrillation electrodes and may be about 5 cm in length.

The atrial/SVC lead shown in FIG. 4 includes elongated insulative lead body 7 carrying three concentric coiled conductors separated from one another by tubular insulative sheaths corresponding to the structure of the ventricular lead. Located adjacent the J-shaped distal end of the lead are ring electrode 9 and extendable helix electrode 13 mounted retractably within an insulative electrode head 15. Each of the electrodes is coupled to one of the coiled conductors within lead body 7. Electrodes 13 and 9 are employed for atrial pacing and for sensing atrial depolarizations. Elongated coil electrode 19 is provided proximal to electrode 9 and coupled to the third conductor within lead body 7. Electrode 19 preferably is 10 cm in length or greater and is configured to extend from the SVC toward the tricuspid valve. In one embodiment of the present invention, approximately 5 cm of the right atrium/SVC electrode is located in the right atrium with the remaining 5 cm located in the SVC. At the proximal end of the lead is bifurcated connector 17 carrying three electrical connectors, each coupled to one of the coiled conductors.

The coronary sinus lead shown in FIG. 4 assumes the form of a coronary sinus lead disclosed in the above cited '838 patent issued to Bardy, and includes elongated insulative lead body 41 carrying one coiled conductor coupled to an elongated coiled defibrillation electrode 21. Electrode 21, illustrated in broken outline in FIG. 4, is located within the coronary sinus and great vein of the heart. At the proximal end of the lead is connector plug 23 carrying an electrical connector coupled to the coiled conductor. The coronary sinus/great vein electrode 41 may be about 5 cm in length.

Implantable PCD 10 is shown in FIG. 4 in combination with leads 1, 7 and 41, and lead connector assemblies 23, 17 and 6 inserted into connector block 12. Optionally, insulation of the outward facing portion of housing 14 of PCD 10 may be provided using a plastic coating such as parylene or silicone rubber, as is employed in some unipolar cardiac pacemakers. The outward facing portion, however, may be left uninsulated or some other division between insulated and uninsulated portions may be employed. The uninsulated portion of housing 14 serves as a subcutaneous defibrillation electrode to defibrillate either the atria or ventricles. Lead configurations other that those shown in FIG. 4 may be practiced in conjunction with the present invention, such as those shown in U.S. Pat. No. 5,690,686 to Min et al., hereby incorporated by reference herein in its entirety.

FIG. 5 is a functional schematic diagram of one embodiment of implantable PCD 10 of the present invention. This diagram should be taken as exemplary of the type of device in which various embodiments of the present invention may be embodied, and not as limiting, as it is believed that the invention may be practiced in a wide variety of device implementations, including cardioverters and defibrillators.

IMD 10 is provided with an electrode system. If the electrode configuration of FIG. 4 is employed, the correspondence to the illustrated electrodes is as follows. Electrode 25 in FIG. 5 includes the uninsulated portion of the housing of PCD 10. Electrodes 25, 15, 21 and 5 are coupled to high voltage output circuit 27, which includes high voltage switches controlled by CV/defib control logic 29 via control bus 31. Switches disposed within circuit 27 determine which electrodes are employed and which electrodes are coupled to the positive and negative terminals of the capacitor bank (which includes capacitors 33 and 35) during delivery of defibrillation pulses.

Electrodes 2 and 3 are located on or in the ventricle and are coupled to the R-wave amplifier 37, which preferably takes the form of an automatic gain controlled amplifier providing an adjustable sensing threshold as a function of the measured R-wave amplitude. A signal is generated on R-out line 39 whenever the signal sensed between electrodes 2 and 3 exceeds the present sensing threshold.

Electrodes 9 and 13 are located on or in the atrium and are coupled to the P-wave amplifier 43, which preferably also takes the form of an automatic gain controlled amplifier providing an adjustable sensing threshold as a function of the measured P-wave amplitude. A signal is generated on P-out line 45 whenever the signal sensed between electrodes 9 and 13 exceeds the present sensing threshold. The general operation of R-wave and P-wave amplifiers 37 and 43 may correspond to that disclosed in U.S. Pat. No. 5,117,824, by Keimel et al., issued Jun. 2, 1992, for “An Apparatus for Monitoring Electrical Physiologic Signals”, hereby incorporated by reference herein in its entirety.

Switch matrix 47 is used to select which of the available electrodes are coupled to wide band (0.5-200 Hz) amplifier 49 for use in digital signal analysis. Selection of electrodes is controlled by the microprocessor 51 via data/address bus 53, which selections may be varied as desired. Signals from the electrodes selected for coupling to bandpass amplifier 49 are provided to multiplexer 55, and thereafter converted to multi-bit digital signals by A/D converter 57, for storage in random access memory 59 under control of direct memory access circuit 61. Microprocessor 51 may employ digital signal analysis techniques to characterize the digitized signals stored in random access memory 59 to recognize and classify the patient's heart rhythm employing any of the numerous signal processing methodologies known to the art.

The remainder of the circuitry is dedicated to the provision of cardiac pacing, cardioversion and defibrillation therapies, and, for purposes of the present invention may correspond to circuitry known to those skilled in the art. The following exemplary apparatus is disclosed for accomplishing pacing, cardioversion and defibrillation functions. Pacer timing/control circuitry 63 preferably includes programmable digital counters which control the basic time intervals associated with DDD, VVI, DVI, VDD, AAI, DDI and other modes of single and dual chamber pacing well known to the art. Circuitry 63 also preferably controls escape intervals associated with anti-tachyarrhythmia pacing in both the atrium and the ventricle, employing any anti-tachyarrhythmia pacing therapies known to the art.

Intervals defined by pacing circuitry 63 include atrial and ventricular pacing escape intervals, the refractory periods during which sensed P-waves and R-waves are ineffective to restart timing of the escape intervals and the pulse widths of the pacing pulses. The durations of these intervals are determined by microprocessor 51, in response to stored data in memory 59 and are communicated to pacing circuitry 63 via address/data bus 53. Pacer circuitry 63 also determines the amplitude of the cardiac pacing pulses under control of microprocessor 51.

During pacing, escape interval counters within pacer timing/control circuitry 63 are reset upon sensing of R-waves and P-waves as indicated by a signals on lines 39 and 45, and in accordance with the selected mode of pacing on time-out trigger generation of pacing pulses by pacer output circuitry 65 and 67, which are coupled to electrodes 9, 13, 2 and 3. Escape interval counters are also reset on generation of pacing pulses and thereby control the basic timing of cardiac pacing functions, including anti-tachyarrhythmia pacing. The durations of the intervals defined by escape interval timers are determined by microprocessor 51 via data/address bus 53. The value of the count present in the escape interval counters when reset by sensed R-waves and P-waves may be used to measure the durations of R-R intervals, P-P intervals, P-R intervals and R-P intervals, which measurements are stored in memory 59 and used to detect the presence of tachyarrhythmias.

Microprocessor 51 most preferably operates as an interrupt driven device, and is responsive to interrupts from pacer timing/control circuitry 63 corresponding to the occurrence sensed P-waves and R-waves and corresponding to the generation of cardiac pacing pulses. Those interrupts are provided via data/address bus 53. Any necessary mathematical calculations to be performed by microprocessor 51 and any updating of the values or intervals controlled by pacer timing/control circuitry 63 take place following such interrupts.

Detection of atrial or ventricular tachyarrhythmias, as employed in the present invention, may correspond to tachyarrhythmia detection algorithms known in the art. For example, the presence of an atrial or ventricular tachyarrhythmia may be confirmed by detecting a sustained series of short R-R or P-P intervals of an average rate indicative of tachyarrhythmia or an unbroken series of short R-R or P-P intervals. The suddenness of onset of the detected high rates, the stability of the high rates, and a number of other factors known in the art may also be measured at this time. Appropriate ventricular tachyarrhythmia detection methodologies measuring such factors are described in U.S. Pat. No. 4,726,380 issued to Vollmann, U.S. Pat. No. 4,880,005 issued to Pless et al. and U.S. Pat. No. 4,830,006 issued to Haluska et al., all incorporated by reference herein, each in its respective entirety. An additional set of tachycardia recognition methodologies is disclosed in the article “Onset and Stability for Ventricular Tachyarrhythmia Detection in an Implantable Pacer-Cardioverter-Defibrillator” by Olson et al., published in Computers in Cardiology, Oct. 7-10, 1986, IEEE Computer Society Press, pages 167-170, also incorporated by reference herein in its entirety. Atrial fibrillation detection methodologies are disclosed in Published PCT application Ser. No. US92/02829, Publication No. WO92/18198, by Adams et al., and in the article “Automatic Tachycardia Recognition”, by Arzbaecher et al., published in PACE, May-June, 1984, pp. 541-547, both of which are incorporated by reference herein in their entireties.

In the event an atrial or ventricular tachyarrhythmia is detected and an anti-tachyarrhythmia pacing regimen is desired, appropriate timing intervals for controlling generation of anti-tachyarrhythmia pacing therapies are loaded from microprocessor 51 into the pacer timing and control circuitry 63, to control the operation of the escape interval counters therein and to define refractory periods during which detection of R-waves and P-waves is ineffective to restart the escape interval counters.

Alternatively, circuitry for controlling the timing and generation of anti-tachycardia pacing pulses as described in U.S. Pat. No. 4,577,633, issued to Berkovits et al. on Mar. 25, 1986, U.S. Pat. No. 4,880,005, issued to Pless et al. on Nov. 14, 1989, U.S. Pat. No. 4,726,380, issued to Vollmann et al. on Feb. 23, 1988 and U.S. Pat. No. 4,587,970, issued to Holley et al. on May 13, 1986, all of which are incorporated herein by reference in their entireties, may also be employed.

In the event that generation of a cardioversion or defibrillation pulse is required, microprocessor 51 may employ an escape interval counter to control timing of such cardioversion and defibrillation pulses, as well as associated refractory periods. In response to the detection of atrial or ventricular fibrillation or tachyarrhythmia requiring a cardioversion pulse, microprocessor 51 activates cardioversion/defibrillation control circuitry 29, which initiates charging of the high voltage capacitors 33 and 35 via charging circuit 69, under the control of high voltage charging control line 71. The voltage on the high voltage capacitors is monitored via VCAP line 73, which is passed through multiplexer 55 and in response to reaching a predetermined value set by microprocessor 51, results in generation of a logic signal on Cap Full (CF) line 77 to terminate charging. Thereafter, timing of the delivery of the defibrillation or cardioversion pulse is controlled by pacer timing/control circuitry 63. Following delivery of the fibrillation or tachycardia therapy microprocessor 51 returns the device to a cardiac pacing mode and awaits the next successive interrupt due to pacing or the occurrence of a sensed atrial or ventricular depolarization.

Several embodiments of appropriate systems for the delivery and synchronization of ventricular cardioversion and defibrillation pulses and for controlling the timing functions related to them are disclosed in U.S. Pat. No. 5,188,105 to Keimel, U.S. Pat. No. 5,269,298 to Adams et al. and U.S. Pat. No. 4,316,472 to Mirowski et al., hereby incorporated by reference herein, each in its respective entirety. Any known cardioversion or defibrillation pulse control circuitry is believed to be usable in conjunction with various embodiments of the present invention, however. For example, circuitry controlling the timing and generation of cardioversion and defibrillation pulses such as that disclosed in U.S. Pat. No. 4,384,585 to Zipes, U.S. Pat. No. 4,949,719 to Pless et al., or U.S. Pat. No. 4,375,817 to Engle et al., all hereby incorporated by reference herein in their entireties, may also be employed.

Continuing to refer to FIG. 5, delivery of cardioversion or defibrillation pulses is accomplished by output circuit 27 under the control of control circuitry 29 via control bus 31. Output circuit 27 determines whether a monophasic or biphasic pulse is delivered, the polarity of the electrodes and which electrodes are involved in delivery of the pulse. Output circuit 27 also includes high voltage switches which control whether electrodes are coupled together during delivery of the pulse. Alternatively, electrodes intended to be coupled together during the pulse may simply be permanently coupled to one another, either exterior to or interior of the device housing, and polarity may similarly be pre-set, as in current implantable defibrillators. An example of output circuitry for delivery of biphasic pulse regimens to multiple electrode systems may be found in the above cited patent issued to Mehra and in U.S. Pat. No. 4,727,877, hereby incorporated by reference herein in its entirety.

An example of circuitry which may be used to control delivery of monophasic pulses is disclosed in U.S. Pat. No. 5,163,427 to Keimel, also incorporated by reference herein in its entirety. Output control circuitry similar to that disclosed in U.S. Pat. No. 4,953,551 to Mehra et al. or U.S. Pat. No. 4,800,883 to Winstrom, both incorporated by reference herein in their entireties, may also be used in conjunction with various embodiments of the present invention to deliver biphasic pulses.

Alternatively, IMD 10 may be an implantable nerve stimulator or muscle stimulator such as that disclosed in U.S. Pat. No. 5,199,428 to Obel et al., U.S. Pat. No. 5,207,218 to Carpentier et al. or U.S. Pat. No. 5,330,507 to Schwartz, or an implantable monitoring device such as that disclosed in U.S. Pat. No. 5,331,966 issued to Bennet et al., all of which are hereby incorporated by reference herein, each in its respective entirety. The present invention is believed to find wide application to any form of implantable electrical device for use in conjunction with electrical leads.

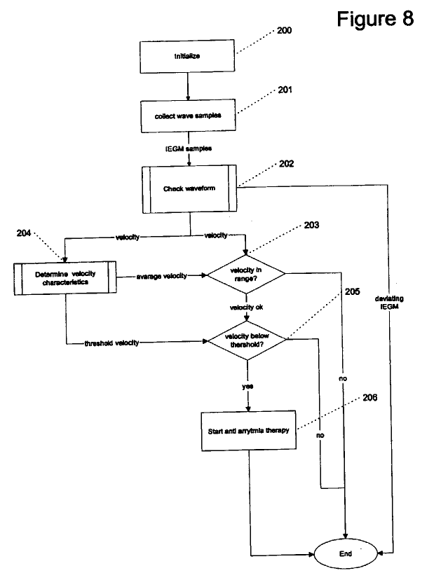

In practicing the method of predicting AF according to this invention a lead 16 having a tip electrode 21 is positioned in the right atrium so that the tip electrode is in place against the interior atrial wall, in a conventional manner. This is illustrated in FIG. 7A, as described above. It is noted that there is no criticality as to exactly where the tip electrode is placed. The lead may be an unipolar lead, in which case the pacemaker case is used as the other electrode; or the lead may have one or more other electrodes, with an electrode displaced significantly proximal to the tip electrode being used as the other electrode so that sensing is effectively unipolar.

Our studies have shown that during normal sinus rhythm the conduction velocity is substantially homogeneous throughout the entire atria. What we want to measure is the deviation in normal conduction velocity, which is substantially the same for the whole atria. The local sensing performed by the tip electrode obtains a signal that is representative of conduction velocity throughout the atria.

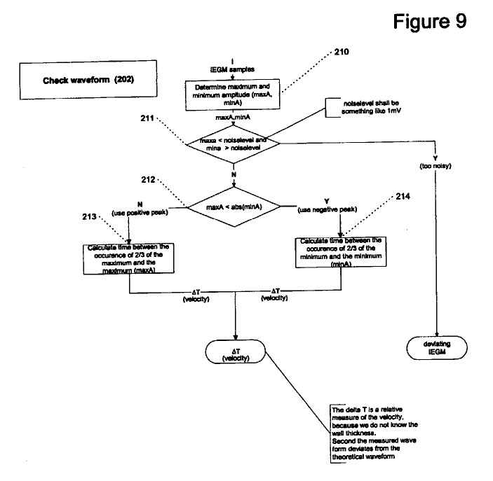

FIG. 8 presents a flow diagram that shows the steps taken in the practice of this invention to monitor conduction velocity. The preferred use is for measuring atrial conduction, in order to detect the onset of AF. However, the same algorithm can be used to detect changes in the right ventricle, using iecg signals sensed from passing ventricular excitation waves (R waves). At 200, the algorithm is initiated. The specific AF prediction algorithm of this invention need not be performed every cycle, but only periodically, e.g., every tenth beat. Changes in conduction velocity occur may occur only over relatively long time periods, such that monitoring conduction velocity for AF need not be done too frequently. However, it is important to detect any change as quickly as possible so as to provide timely therapy. Consequently, the time period for initiating the prediction algorithm of FIG. 8 is a matter of choice, and may be subject to knowledge of the patient's past history. Also at 200 the pacemaker checks to see that conditions are appropriate for the test, e.g., the atrial rate is nominal and stable. If rate is not stable, then changes in the width of the P-wave might not suggest changes in conduction velocity.

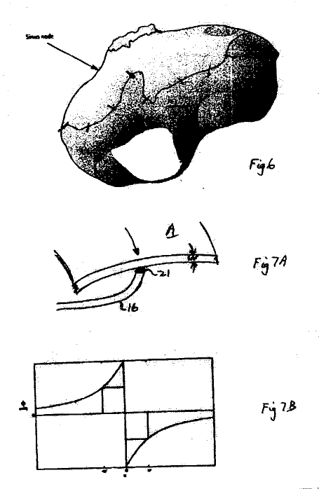

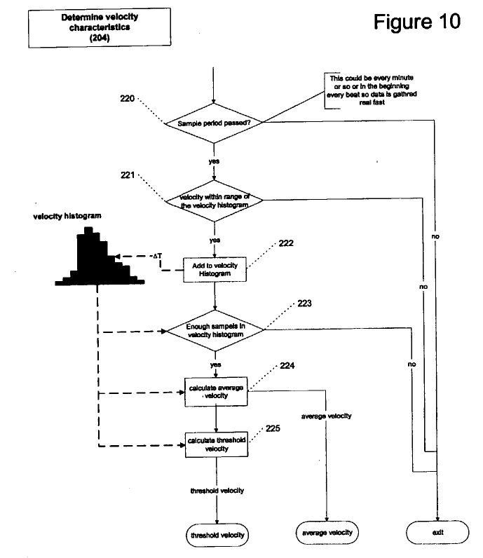

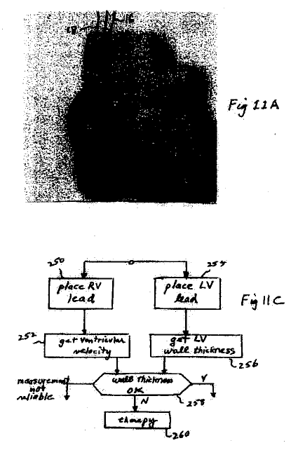

As the excitation wave depicted in FIG. 6 advances over the entire atria it proceeds from the sinus node through to the last activated area located below the inferior pulmonary vein on the left lateral wall. For normal pacemaker operation, the P-wave is detected and processed every cycle, for control purposes. Samples of the sensed excitation wave, or intracardial P-wave are sensed and collected as shown at block 201. Generally, a plurality of P-wave samples is taken at each monitoring episode. For example, n consecutive IEGM (P-wave) samples may be collected for the purpose of the AF prediction algorithm. At 202, pertinent P-wave characteristics are determined. In the embodiment of FIG. 8, the maximum and minimum amplitudes are determined by checking the waveform. In a preferred embodiment, the duration is determined as the Δt from ⅔ of maximum amplitude of the rising slope to that of the falling wave slope, as discussed in more detail below in connection with FIG. 9. Other formulae can be employed to obtain a measure of the wave duration Δt within the scope of the invention. The morphology of the P-waves may also be checked, to determine that they reflect normal physiological events. If the waveforms are found to be deviating, the samples must be rejected, and the routine exits. However, if they are nominal, then the routine continues. The “velocity” measure that is provided from the operation at 202 is a relative velocity, as the atrial wall thickness is not known. Further, the waveform will deviate somewhat from the theoretical. At 204, the velocity samples from the operation at 202 are stored, and a subroutine (FIG. 10) is employed to obtain certain velocity characteristics. The wave widths, representing relative velocity values, are suitably averaged to obtain a value V representative of average conduction velocity for the patient. Additionally, a velocity threshold is calculated, for subsequent use in determining whether a change in conduction velocity is indicative of AF (or VF, in a ventricular embodiment).

At block 203, the routine compares the current velocity with the average velocity to determine if it is within a proper range. If the current velocity reading deviates significantly from the prior range, the data is considered not good, and the routine exits. However, if the velocity measure is ok, then the velocity is compared to the threshold velocity at 205. If velocity V is not below the threshold, the routine concludes that AF is not predicted, and exits. If velocity is below threshold, then at 206 the system initiates a suitable anti-arrhythmia therapy.

FIG. 9 illustrates a suitable “check waveform” subroutine, corresponding to block 202 of FIG. 8. At block 210, the maximum (positive) and minimum (negative) amplitudes are determined for each sensed waveform. The values, shown as maxA and minA respectively, are compared at 211 to a predetermined noise level. Noise level may be set at, for example, about 1 mV. If neither amplitude is greater than the noise level, the conclusion is that the data is too noisy, and the routine exits. However, if this is not the case, then at 212 the routine compares maxA and the absolute value of minA, and picks the largest of the two values, i.e., determines whether to use the positive or negative peak. For the positive peak, at 213 Δt is calculated as the time between ⅔ of the maximum of the rising slope and ⅔ of the maximum of the falling slope. Of course, other criteria for obtaining a measure of the duration value Δt can be employed within the scope of the invention, the specific measure being a matter of choice. Likewise, at 214 a value of Δt is obtained from the negative peak, if the negative peak has been chosen. In either event, the product of the subroutine is a value of Δt for each analyzed waveform, which value represents relative velocity for the conduction velocity embodiment of this invention.

FIG. 10 provides a flow diagram of block 204, detailing steps taken to determine certain velocity characteristics for the velocity determination embodiments. At 220, it is determined whether a predetermined sample period has passed. The sample period is a variable, and could be, e.g., every minute. However, at start up of the pacemaker system, this could be set to operate on every beat, so as to build up a database quickly. If the sample period has passed, the routine then goes to step 221 and determines whether the velocity of each waveform is within the range of a stored velocity histogram, i.e., whether it is a value that is reasonable and can be accepted. If the velocity data is not within the range, the routine exits; if yes, it goes to block 222 and adds each sample to a velocity histogram that is continually built in memory. At 223, the routine determines whether there are enough samples in the velocity histogram to provide a good statistical basis. Thus, at start up following implant, or when turning on the embodiment by external programming, it is necessary to wait to get a good database. When this has been accomplished, at 224 the routine calculates an average (relative) velocity. Then, at 225, a threshold velocity is calculated. This may be done just once, when the histogram has sufficient samples; or it may be repeated a predetermined number of times following start up. Threshold is a figure that optionally may be initially programmed by the physician. However, it preferably is calculated as a function of initial average velocity. For example, threshold may be calculated as Vavg+X % of Vavg, where X is a programmable value in the range of 20-50. Other methods of determining the threshold value may be used within the scope of the invention.

It is noted that determination of onset of AF can be done in other ways than as depicted and described in connection with block 205 in FIG. 8. Thus, rather than calculating a threshold, the algorithm may look for a predetermined decrease in the measure of velocity over a predetermined relatively long time period, e.g., a 25% decrease over a month. Further, depending on patient history, the response may be to simply post a flag that indicates onset of AF, so that at a subsequent confirmation an AF therapy will be commenced. Or, the response can be to immediately initiate a relatively high rate pacing sequence to attempt to break up the dangerous condition. Anti-AF pacing therapies are well known in the art, and the invention embraces any suitable such pacing therapy.

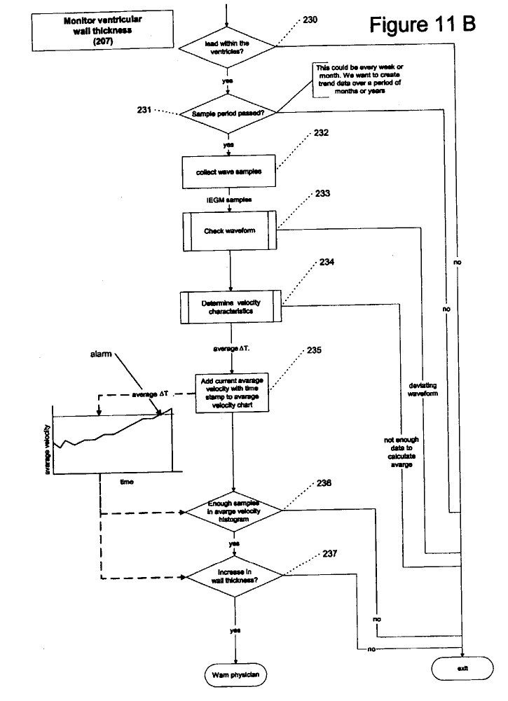

The embodiment of this invention for determining ventricular wall thickness is shown in FIGS. 11A,B and C. FIG. 1A illustrates the placement of a lead 150 having a tip electrode 151 that is positioned adjacent to the left free ventricular wall. The lead is passed through the coronary sinus to an epicardial vewin on the free wall of the left ventricle, in the same manner as is done for bi-ventricular pacing systems. The tip 151 detects the repolarization wave (R wave) as it passes by the tip, in the same manner as described above for the atrial wave. The pacemaker case is suitably used as the indifferent electrode. A second electrode for pacing may be positioned in normal fashion in the right ventricle, substantially at the apex. The R-waves sensed by the left ventricular tip electrode (R_{L}) are processed in the same manner as done in the AF predictor algorithm. Concurrently, R-waves sensed by the right ventricular electrode (R_{R}) are examined to verify that overall there has been no significant change in ventricular conduction velocity. Having verified this, then the changes in width (Δt) of the wave sensed by the left ventricular electrode can be interpreted as changes in ventricular wall width. As discussed above and as illustrated in FIG. 1B, the longer the wave duration (Δt) the greater the wall thickness. In this embodiment, changing values of relative wall thickness are compared with a wall threshold value, and appropriate pacing therapy or an adjustment in medication is initiated when the threshold is exceeded.

FIG. 11B shows an exemplary flow diagram of an algorithm for determining the wall thickness of the free wall of the left ventricle. Such determination can be very useful for assessing hypertrophy of the ventricle, so that appropriate therapy can be undertaken. As discussed above, measurement of ventricular wall thickness is undertaken with the assumption that conduction velocity in the left ventricle is substantially constant, and that a significant decrease in the width (Δt) of the sensed R wave is due to enlargement of the left wall thickness.

As illustrated in FIG. 11B, at 230 it is determined that a lead is properly placed within the left ventricle or adjacent to the left ventricular wall (as illustrated in FIG. 11C). Assuming that this condition is met, at 231 the routine tests to see that a programmable sample period has passed. If not, the routine exits, but if yes, it goes on to block 232 and collects wave samples in the same manner as discussed in connection with the routine of FIG. 8. The iegm samples are checked for waveform at 233, according to the subroutine disclosed in FIG. 9, yielding representations of Δt that, in this case, are measures of relative left ventricular wall thickness. The steps taken in block 233 may also include a check to determine that the wall thickness measure is within an acceptable range, corresponding to step 203 in FIG. 8. The processing at block 233 produces duration measures that are sent for processing at block 234.

Block 234 performs the same operations as shown in FIG. 10, i.e., the same “determine velocity characteristics” operations are carried out. However, in this embodiment the Δt values represent relative left ventricular wall thickness and values of average Δt are calculated. At 235 the average Δt_{avg} values are added, along with a time stamp, to a histogram in the same manner as illustrated for the velocity data in FIG. 10. At 236, the routine determines whether enough samples have been placed in the histogram. If not, the routine exits until a sufficient database has been built. If there is enough data, then at 237 the routine determines whether or not there has been a significant increase in left ventricular wall thickness. This may be done in a chosen one of several ways. For example, the average Δt value may be compared to a predetermined alarm value and if it exceeds the alarm value, then a warning is posted to the physician. The warning can be in any suitable form, such as by setting a flag that is read whenever the physician next checks the device. Alternatively, the determination can be made by looking for a given percentage increase in relative wall thickness over a period of time, e.g., a relatively long period of time such as a 1-6 months, or even annually. Since wall thickness does not change rapidly, this algorithm need only be entered once a week, for example. Further, the physician will be interested in long term trend data.

In both this embodiment and the atrial velocity embodiment, heart rate may be checked to make sure that it is stable, and if not stable the algorithm exits.

Another embodiment is illustrated by the flow diagram of FIG. 11C. In this embodiment, RR waves are collected from the right ventricle and used to determine whether ventricular conduction velocity has remained substantially constant. This is done by examining the Δt of sensed RR waves in the same manner as described above for atrial waves, to ensure that the right R wave signal has maintained a substantially constant duration. A substantially constant duration of RR waves provides confirmation of stable ventricular conduction velocity, such that changes in the measurement of wave duration at the left ventricular wall can be reliably taken as indicating change in wall thickness.

As shown in FIG. 11C, at 250 a lead 18 is placed in the right ventricle, having a tip electrode 29. For purposes of measuring conduction velocity, the tip electrode may be positioned other than at the apex. At 252, a measure of ventricular conduction velocity is obtained, using the same flow diagram as in FIG. 8; the velocity threshold is set to indicate whether or not there has been a significant change in velocity. A lead 150 is placed so that the tip 151 is adjacent the free wall of the left ventricle, as indicated at block 254. At 256, the left ventricular wall thickness is obtained, as per FIG. 11B. At 258, the algorithm checks the ventricular velocity to determine if it is stable. If not, the routine exits, as the wall thickness measure is then not accurate. If velocity is stable, then the routine determines if the wall thickness is OK, e.g., has the increase exceeded a predetermined limit? If wall thickness is OK, the routine exits; if not, then therapy is applied, as indicated at 260.

It is noted that the invention can be practiced with various combinations of determining conduction velocity and wall thickness. As illustrated in FIG. 11A, the invention comprises a multi-chamber embodiment, where either or both ventricular and atrial conduction velocity can be determined, and left ventricular wall thickness can be determined. The choice of functions can be programmable, in accord with the current state of the art.

The present invention is not limited to any particular combination of hardware and software per se, but may find further application with any form of software supplementing hardware. For example, other software embodiments that achieve the ability to efficiently store and manipulate the data, and analyze the sensed depolarization signals, are within the scope of the invention.

In the claims, means-plus-function clauses are intended to cover the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts a nail and a screw are equivalent structures. As indicated above, in the claims the terms pacemaker and pacemaker system include all of the different types of pacing devices, as well as cardioverters and defibrillators.

What is claimed is:

1. An implantable cardiac treatment system for predicting the onset of an atrial arrhythmia, comprising a pacemaker device and a lead,
said lead having a tip electrode adapted to be positioned against the myocardial wall of the patient's right atrium to sense P-waves and having conducting means for conducting sensed P-waves,
said pacemaker device comprising:
receiving means for receiving said sensed P-waves from said lead conducting means;
processing means for processing sensed P-waves to obtain a measure of the P-wave signal width of each processed P-wave;
monitoring means for monitoring said width measures over a long period to determine the onset of atrial fibrillation (AF); and
response means for responding to a determination of onset of AF by providing a predetermined AF pacing therapy.

2. The system as described in claim 1, wherein said processing means comprises determining means for operating on a P-wave to determine the signal width in milliseconds between two predetermined reference points on said P-wave.

3. The system as described in claim 2, wherein said determining means comprises amplitude means for determining the maximum amplitude of a processed P-wave signal and reference means for setting said reference points relative to said maximum amplitude.

4. The system as described in claim 3, wherein said reference means comprises means for setting said reference points where the leading and trailing edges of said P-wave are a predetermined fraction below said maximum amplitude.

5. The system as described in claim 3, wherein said amplitude means further comprises minimum means for determining the minimum amplitude of a processed P-wave, and said reference means comprises means for setting said reference points where the leading and trailing edges of said P-wave are a predetermined distance between said maximum and minimum amplitudes.

6. The system as described in claim 1, comprising storage means for storing data representative of said width measures over said long period.

7. The system as described in claim 6, wherein said response means comprises means for evaluating said stored data and for determining the presence or absence of AF onset as a function of said evaluated data.

8. The system as described in claim 6, wherein said response means comprises means for evaluating said stored data over a period of at least a week and for determining the presence or absence of AF onset as a function of said evaluated data.

9. The system as described in claim 6, comprising evaluating means for evaluating said stored data to determine when said width measure has increased by a predetermined percentage.

10. A system for continuously determining a characteristic of a depolarization wave in a wall of a patient's heart and for initiating a pacing response to the heart as a function of said determining, comprising:
signal means for obtaining a signal representative of the passage of a depolarization wave past a point on said wall;
Δt means for determining a measure of the signal width of the depolarization wave from each said obtained signal;
analyzing means for analyzing said signal width measures and for determining when said time width measures indicate a dangerous condition in said heart; and
response means for initiating a pacing therapy in response to a said dangerous condition.

11. The system as described in claim 10, wherein said signal means comprises a tip electrode positioned against the right atrial wall of said heart.

12. The system as described in claim 10, wherein said analyzing means comprises velocity means for determining conduction velocity in said heart.

13. The system as described in claim 12, wherein said velocity means comprises criteria means for comparing said signal width measures with predetermined criteria.

14. The system as described in claim 10, wherein said signal means comprises a tip electrode positioned adjacent to the patient's left free ventricular wall.

15. The system as described in claim 14, wherein said analyzing means comprises wall thickness means for determining from said signal width measures when the patient's left free ventricular wall has thickened to a dangerous value.

16. The system as described in claim 15, wherein said wall thickness means comprises criteria means for comparing said signal width measures with predetermined criteria.

17. The system as described in claim 10, wherein said signal means further comprises stability means for determining whether said signals are stable.

18. The system as described in claim 17, comprising rate means for limiting the operation of said system to conditions where the cardiac rate is within a predetermined range.

19. The system as described in claim 11, comprising threshold means for determining a velocity threshold, comparison means for comparing said conduction velocity with said threshold, and wherein said response means is enabled when conduction velocity is determined to be slower than said threshold.

20. The system as described in claim 19, wherein said threshold means comprises set means for setting said threshold as a function of signal width measures.

21. The system as described in claim 20, wherein said threshold means comprises programmable threshold criteria.

22. A method of determining a potentially dangerous condition of a patient's heart, comprising:
positioning a tip electrode against the inside wall of a chamber of said heart;
obtaining from said tip electrode wave duration signals representative of the time width of passing depolarization waves, and storing data from said signals;
processing said signals to determine a significant change in the time width of said passing depolarization waves; and
indicating a said potentially dangerous condition when a said significant change is determined.

23. The method as described in claim 22, comprising positioning a lead with a tip electrode within the patient's right atrium, and obtaining wave duration signals representative of the time width of depolarization waves in the right atrium as they pass said tip electrode.

24. The method as described in claim 23, wherein said processing comprises checking the sensed waveforms to determine that they are distinguished from noise.

25. The method as described in claim 24, wherein said processing comprises storing data representative of waveform duration in velocity categories, and comparing sensed data with said stored data to determine that said sensed data is within the range of said stored data.

26. The method as described in claim 25, comprising calculating from said stored data values representative of average conduction velocity, and comparing said average velocity values with predetermined criteria.

27. The method as described in claim 26, comprising determining a velocity threshold, and comparing each value of average conduction velocity with said threshold.

28. The method as described in claim 27, comprising determining when said average conduction velocity is less than said threshold, and initiating therapy in response to a said determination.

29. The method as described in claim 22, comprising positioning a tip electrode adjacent to the patient's left free ventricular wall, and obtaining waveforms of passing ventricular depolarization waves.

30. The method as described in claim 29, comprising processing said obtained waveforms to obtain a measure of the duration of each, and storing data representative of said duration measures.

31. The method as described in claim 30, comprising storing time information with said stored duration data.

32. The method as described in claim 31, comprising comparing said duration data with predetermined criteria.

33. The method as described in claim 32, wherein said criteria comprises a predetermined value, and indicating a significant increase in left ventricular wall thickness when said duration measure is greater than said predetermined value.

34. The method as described in claim 29, comprising placing a lead with a tip electrode within the patient's right ventricle and positioning said tip electrode against the ventricular wall, and obtaining reference depolarization waveforms from said right ventricle.

35. The method as described in claim 34, comprising determining from said reference waveforms whether ventricular conduction velocity has changed significantly and verifying an indication of significant increase in left ventricular wall thickness when said ventricular conduction velocity has not changed significantly.

36. The method as described in claim 35, comprising initiating therapy in response to an indication of increase in ventricular wall thickness.

37. An implantable system for obtaining a measure of relative conduction velocity in a patient's atria, comprising:
a lead that carries a tip electrode, adapted to be positioned within the patient's right atrium with said tip electrode snug against the inside wall of said atrium;
sensing means for obtaining from said tip electrode wave samples of depolarization waves that pass said tip electrode;
processing means for processing said wave samples to obtain measures of relative conduction velocity; and
analyzing means for analyzing said measures to provide an indication of change in conduction velocity in said patient's atria.

38. The system as described in claim 37, wherein said processing means comprises duration means for obtaining for each said waveform a measure of the duration of said waveform.

39. The system as described in claim 38, wherein said duration means comprises means for determining a maximum value of the waveform, and measuring means for measuring said duration as the time duration between points on the waveform between values that are a predetermined percentage of said maximum value.

40. The system as described in claim 39, comprising storage means for storing data representative of said duration measures.

41. The system as described in claim 40, comprising threshold means for determining from said stored data a threshold value representative of a conduction velocity threshold.

42. The system as described in claim 41, comprising current means for determining a measure of current conduction velocity, and comparing means for comparing said current measure with said threshold value.

43. The system as described in claim 37, comprising initial means for determining and storing a measure of initial relative conduction velocity.

44. The system as described in claim 43, comprising current means for determining a measure of current conduction velocity from collected waveforms, and comparing means for comparing said current measure with said initial measure.

45. The system as described in claim 37, comprising checking means for checking the reliability of said wave samples in terms of indicating relative conduction velocity.

46. The system as described in claim 45, wherein said checking means comprises noise comparison means for determining that said waveforms are of significant amplitude above the prevailing noise level.

47. The system as described in claim 46, wherein said checking means comprises rate means for determining that the rate of said wave samples is within a predetermined range.

48. The system as described in claim 37, comprising sample means for determining that the collected number of waveform samples is sufficient to provide an accurate result.

49. A multi-chamber pacing system having leads postionable in a patient's heart, comprising:
wall thickness means for obtaining a measure of left ventricular wall thickness;
wall analysis means for determining when the patient's left ventricular wall has thickened unacceptably;
atrial velocity means for obtaining a measure of atrial conduction velocity; and
atrial velocity analysis means for determining when the patient's atrial conduction velocity has slowed unacceptably.

50. The system as described in claim 49, further comprising ventricular velocity means for obtaining a measure of ventricular conduction velocity, and ventricular velocity analysis means for analyzing changes in ventricular conduction velocity.
